(19) United States
(12) Patent Application Publication
(10) Pub. No.: US 2024/0003914 A1
(43) Pub. Date: Jan. 4, 2024

(54) NICOTINAMIDE DINUCLEOTIDE (NAD) DETECTION AND QUANTIFICATION

(71) Applicants: THE SCRIPPS RESEARCH INSTITUTE, La Jolla, CA (US); VOVA IDA THERAPEUTICS, INC., Jupiter, FL (US)

(72) Inventors: Corinne LASMÉZAS, Palm Beach Gardens, FL (US); Minghai ZHOU, Jupiter, FL (US)

(21) Appl. No.: 18/252,798

(22) PCT Filed: Nov. 12, 2021
(86) PCT No.: PCT/US21/59200

§ 371 Date: May 12, 2023

Related U.S. Application Data
(60) Provisional application No. 63/113,049, filed on Nov. 12, 2020.

(51) Int. Cl. G01N 33/68; A61K 31/455; A61P 3/00

(52) U.S. Cl. CPC G01N 33/6893; A61K 31/455; A61P 3/00

(57) ABSTRACT

Methods of preventing disease or treating a subject likely to develop or having a neurodegenerative, degenerative or metabolic disease include administration of nicotinamide dinucleotide (NAD) modulating agents. Compositions include one or more agents which increase or decrease NAD in vivo or in vitro.

### CROSS-REFERENCES TO RELATED APPLICATIONS

This application claims the benefit of priority of U.S. Provisional Application No. 63/113,049 filed on Nov. 12, 2020, which is incorporated herein by reference in its entirety and for all purposes.

### STATEMENT REGARDING FEDERALLY SPONSORED RESEARCH

This invention was made with government support under Grant Number R01NS103195 awarded by the National Institutes of Health. The government has certain rights in the invention.

### FIELD

The present disclosure relates to assays for NAD detection and quantification and their use as diagnostic tools, for monitoring disease progression or for monitoring the efficacy of treatment of a disease.

### BACKGROUND

Alzheimer's and Parkinson's diseases (AD, PD), prion diseases and amyotrophic lateral sclerosis (ALS) share a common pathogenic mechanism, i.e. the misfolding and aggregation of endogenous proteins that become toxic for neurons. PD and ALS are heterogeneous diseases from a clinical, genetic and molecular standpoint. In ALS, clinical heterogeneity manifests itself in terms of the site of onset (bulbar vs spinal), relative degree of upper and lower motor neuron involvement and progression rate[1]. Moreover, about 50% of patients will suffer from extra-motor manifestations to some degree in addition to their motor symptoms[2]. Genetic heterogeneity exists for both sALS and fALS, with more than genes identified conferring susceptibility or causation[3]. Mutated genes include: superoxide dismutase 1 (SOD1), C9ORF72, TAR DNA Binding Protein (TARDBP, encoding the protein TDP-43), Fused in sarcoma (FUS)[3-6]. In PD, clinical and genetic heterogeneity are no less. Clinical diagnosis of PD requires manifestation of bradykinesia and at least one of the following: resting tremor, muscular rigidity and postural reflex impairment. In certain PD patients, tremors constitute the predominant symptom, whereas in others rigidity and/or postural instability prevail. In addition, a range of other symptoms are observed in up to 50% of PD patients: sialorrhoea, constipation, depression, psychiatric manifestations, cognitive dysfunction. Familial PD cases have been linked to mutations in five genes, SNCA, GBA, PARK2/Parkin, PINK1, DJ-1, LRRK2, while an increasing number of other genes are found to be linked to PD risk[7-12].

Since 2007, over 80 phase 2 or 3 clinical trials have been conducted for ALS leading to only one drug approval in certain countries (edaravone)[13]. Low success rate of ALS clinical trials has been attributed to uncertain biological targets, late intervention and lack of strategies to address clinical heterogeneity[13]. Consequently, considerable effort is being devoted to improving patient stratification in clinical trials and developing markers for treatment efficacy. Prognostic models based on predefined sources of heterogeneity (including but not limited to age at onset, forced vital capacity, diagnostic delay, clinical rating slope, bulbar onset) have been developed[1] and applied for patient stratification based on individual risk; however it has been shown to reduce the eligibility rate by up to 60%[14, 15], underscoring the need for predictive enrichment to reduce heterogeneity and select patients most likely to respond to a treatment[13]. Given the pressing need, several biomarkers are currently being evaluated for the diagnosis and prognosis of ALS. Serum/plasma and cerebrospinal fluid (CSF) neurofilament light (NfL) and phosphorylated neurofilament heavy (pNfH) were found to be potential pharmacodynamic biomarkers[16, 17], with NfL being prognostic of the Revised ALS Functional Rating Scale (ALSFRS-R) slope and survival in sporadic but not familial ALS patients[16, 18]. The pharmacodynamic utility of NfL and pNfH is to be demonstrated in phase 2 trials, with the caveat that it will only be confirmed once paired with an effective treatment for ALS[16]. Urinary extracellular neurotrophin receptor p75 (p75^{NTR}ECD or p75^{ECD}) is a biomarker for ALS disease progression both in patients and Tg(SOD1*G93A) mice[19, 20], proposed for pharmacodynamic application[21]. A meta-analysis study revealed that CSF TDP-43 could be a promising biomarker for ALS[22]. Other potential biomarkers are: cytokine panels[17, 23], serum creatinine, albumin, C-reactive protein and glucose[24], inflammatory biomarkers and miRNAs[25] including TDP-43 binding miR-132 downregulated in sporadic and a subset of familial ALS[26].

In PD, one strategy to reduce heterogeneity consists in enrolling “de novo” patients, i.e. patients who have not been treated before and are typically in very early stages of disease[27, 28]. However, such patients are difficult to enroll in clinical trials, and these trials don't inform on the efficiency of the treatment for clinically more advanced patients. The use of DaTscan also helps reduce heterogeneity and changes from baseline may be used to measure disease progression[29]. However, DaTscan requires expensive imaging equipment and increases the costs of clinical trials and exposes the patient to radiation. Blood and CSF α-synuclein species as well as blood NfL are also under investigation as biomarkers for disease progression in PD[30].

In AD, tau PET imaging is being used to assess progression of clinically relevant neuropathological lesions but has the same disadvantages as DaTscan[31, 32]. Decreases in cerebrospinal fluid (CSF) concentrations of amyloid-beta 42 (A1342), concentration ratio of CSF Aβ42 to Aβ40, and elevation of CSF phophorylated tau species (markers of axonal damage and neurofibrillary tangles) are useful biomarkers to improve the accuracy of AD diagnosis[33]. Recently, blood p181tau and p217 tau were shown to be elevated in blood of AD patients compared with healthy controls and other neurodegenerative conditions and may be useful to screen for tau pathology associated with AD[34, 35].

### SUMMARY

In ALS, AD, PD and other neurodegenerative diseases, an easily accessible biomarker is needed. Ideally, such a biomarker would help address clinical heterogeneity by providing a means for patient stratification upon enrollment and would be predictive of a positive clinical outcome in an interventional clinical trial. Further, it would be used as a pharmacodynamic marker to monitor the beneficial effect of a neuroprotective treatment under investigation in a clinical trial, and as a surrogate marker for treatment efficiency. It could also be used by neurologists to monitor disease progression, treatment compliance and treatment efficacy. Accordingly, provided herein is the detection and quantification of a biomarker, nicotinamide dinucleotide (NAD), in animals suffering from a neurodegenerative disease. Further, quantification of NAD provides a means for patient stratification and an objective quantifiable measure of treatment efficacy, thereby accelerating the demonstration that a drug candidate has utility.

In an aspect provided is a method of detecting and quantifying nicotinamide dinucleotide (NAD) in a biological sample. The method includes: protecting NAD in a biological sample using an inhibitor of NAD degradation; optionally disrupting the biological sample; optionally clarifying a biological sample; optionally fractionating the biological sample; collecting the fractionated biological sample; and measuring NAD, thereby, detecting and quantifying nicotinamide dinucleotide (NAD) in the biological sample.

In certain embodiments, the inhibitor of NAD degradation is a metabolite, a chemical reagent, a chemical compound, a lipid, a polymer, a nucleic acid or a protein. In certain embodiments, the inhibitor of NAD degradateion is nicotinamide.

In certain embodiments, the addition of the inhibitor of NAD degradation occurs at the same time as diluting the biological sample.

In certain embodiments, the biological sample is disrupted by mechanical or chemical lysis and/or sonication. In certain embodiments, the biological sample is clarified by centrifugation and/or filtration.

In certain embodiments, the fractionating is performed by filtration and/or chromatography. In certain embodiments, the fractionated biological sample comprises a fraction cut of about 100 kDa, 50 kDa, 30 kDa, 10 kDa, 5 kDa or 3 kDa. In certain embodiments, the fractionating removes one or more factors interfering with NAD detection.

In certain embodiments, the measuring the NAD comprises measuring and quantifying NAD metabolite levels using an enzymatic cycling assay.

In an aspect, provided is a method of preventing disease or treating a subject likely to develop or having a neurodegenerative, degenerative or metabolic disease. The method includes: diagnosing the subject; and administering to the subject a therapeutic agent, thereby, preventing disease or treating the subject having a neurodegenerative disease.

In certain embodiments, the neurodegenerative disease includes Alzheimer's disease (AD), Parkinson's diseases (PD), dementia with Lewy Bodies (DLB), Fronto-temporal dementia (FTD), chronic traumatic encephalopathy (CTE), prion diseases, Huntington's disease (HD), amyotrophic lateral sclerosis (ALS) or multiple sclerosis (MS).

In certain embodiments, the metabolic disease comprises disease is diabetes or non-alcoholic fatty liver disease (NAFLD).

In certain embodiments, the pharmacological effect of the treatment is monitored by quantification of nicotinamide dinucleotide (NAD) in blood, brain, cerebrospinal fluid, another biological fluid or a tissue.

In certain embodiments, a decrease or increase in NAD as compared to a normal control, is diagnostic of a neurodegenerative, degenerative or metabolic disease.

In certain embodiments, detection of an increase or decrease in NAD or normalization of NAD levels as compared to a normal control, is indicative of therapeutic efficacy of the treatment.

In an aspect, provided is a method of identifying candidate neuroprotective or disease-modifying therapeutic agents comprising contacting a cell or biological sample with a candidate agent, measuring the nicotinamide dinucleotide (NAD) levels in the cell or biological samples as compared to controls and determining whether NAD is modulated in response to the agent.

In certain embodiments, an increase in NAD levels as compared to a control is indicative of a neuroprotective and/or disease-modifying therapeutic agent.

In certain embodiments, the cells include cells obtained from a subject diagnosed with a neurodegenerative, degenerative or metabolic disorder, cell lines, transfected cells, mesenchymal cells, induced pluripotent cells (iPSCs), stem cells or combinations thereof.

In certain embodiments, the assay is a high-throughput screening assay.

Other aspects of the invention are disclosed infra.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element. Thus, recitation of “a cell”, for example, includes a plurality of the cells of the same type. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

“About” as used herein when referring to a measurable value such as an amount, a temporal duration, and the like, is meant to encompass variations of +/−20%, +/−10%, +/−5%, +/−1%, or +/−0.1% from the specified value, as such variations are appropriate to perform the disclosed methods. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

The term “agent” is used to describe a compound that has or may have a therapeutic or pharmacological activity. Agents include compounds that are known drugs, compounds for which therapeutic activity has been identified but which are undergoing further therapeutic evaluation, and compounds that are members of collections and libraries that are to be screened for a pharmacological activity.

The term “assay” used herein, whether in the singular or plural shall not be misconstrued or limited as being directed to only one assay with specific steps but shall also include, without limitation any further steps, materials, various iterations, alternatives etc., that can also be used. Thus, if the term “assay” is used in the singular, it is merely for illustrative purposes.

As used herein, “biological samples” include solid and body fluid samples. The biological samples used in the present invention can include cells, protein or membrane extracts of cells, extracellular vesicle extracts, exosomal extracts, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

As used herein, the term “biomarker” refers to a molecule that is associated either quantitatively or qualitatively with a biological change. Examples of biomarkers include polypeptides, proteins or fragments of a polypeptide or protein; and polynucleotides, such as a gene product, RNA or RNA fragment; and other body metabolites. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test).

The term “high-throughput screening” or “HTS” refers to a method drawing on different technologies and disciplines, for example, optics, chemistry, biology or image analysis to permit rapid, highly parallel biological research and drug discovery. HTS methods are known in the art and they are generally performed in multiwell plates with automated liquid handling and detection equipment; however it is envisioned that the methods of the invention may be practiced on a microarray or in a microfluidic system.

A “metabolic disease” refers to any of the diseases or disorders that disrupt normal metabolism, the process of converting food to energy on a cellular level. Thousands of enzymes participating in numerous interdependent metabolic pathways carry out this process. Metabolic diseases affect the ability of the cell to perform critical biochemical reactions that involve the processing or transport of proteins (amino acids), carbohydrates (sugars and starches), or lipids (fatty acids). Metabolic diseases are typically hereditary, yet most persons affected by them may appear healthy for days, months, or even years. The onset of symptoms usually occurs when the body's metabolism comes under stress—for example, after prolonged fasting or during a febrile illness. For some metabolic disorders, it is possible to obtain prenatal diagnostic screening. Such analysis usually is offered to families who have previously had a child with a metabolic disease or who are in a defined ethnic group. For example, testing for Tay-Sachs disease is relatively common in the Ashkenazi Jewish population. Countries that perform screening for metabolic diseases at birth typically test for up to 10 different conditions. Tandem mass-spectrometry is a one technology that allows for the detection of multiple abnormal metabolites almost simultaneously, making it possible to add approximately 30 disorders to the list of conditions for which newborns may be tested. If an infant is known to have a metabolic disorder soon after birth, appropriate therapy can be started early, which may result in a better prognosis.

A “neurological disease” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Drager syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g. of the CNS and/or brain, including brain metastases resulting from cancer elsewhere in the body), multiple sclerosis.

A “neuroprotective agent” is a drug or therapeutic agent that treats one or more neurological disorder(s). Neurological disorder drugs of the invention include, but are not limited to, small molecule compounds, antibodies, peptides, proteins, natural ligands of one or more CNS target(s), modified versions of natural ligands of one or more CNS target(s), aptamers, inhibitory nucleic acids (i.e., small inhibitory RNAs (siRNA) and short hairpin RNAs (shRNA)), ribozymes, and small molecules, or active fragments of any of the foregoing. Exemplary neuroprotective agents include, but are not limited to: antibodies, aptamers, proteins, peptides, inhibitory nucleic acids, gene therapy constructs and their vector and small molecules and active fragments of any of the foregoing that either are themselves or specifically recognize and/or act upon (i.e., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the corresponding disorders they may be used to treat: Brain-derived neurotrophic factor (BDNF), Chronic brain injury (Neurogenesis), Fibroblast growth factor 2 (FGF-2), Anti-Epidermal Growth Factor Receptor Brain cancer, (EGFR)-antibody, Glial cell-line derived neural factor Parkinson's disease, (GDNF), Brain-derived neurotrophic factor (BDNF) Amyotrophic lateral sclerosis, depression, Lysosomal enzyme Lysosomal storage disorders of the brain, Ciliary neurotrophic factor (CNTF) Amyotrophic lateral sclerosis, Neuregulin-1 Schizophrenia, Anti-HER2 antibody (e.g. trastuzumab) Brain metastasis from HER2-positive cancer.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Any compositions or methods provided herein can be combined with one or more of any of the other compositions and methods provided herein.

### BRIEF DESCRIPTION OF THE DRAWINGS

FIGS. 1A-1J are a series of graphs demonstrating the signal linearity during the NAD detection window with the optimized method, and examples of method development steps.

FIG. 1A shows NAD standard (25 nM in PBS, 1 mM NAM, pH=7.4).

FIG. 1B shows that One mouse brain hemisphere is homogenized on ice in 500 μl PBS (pH=7.4) in the presence of 1 mM nicotinamide (NAM) to inhibit NAD consuming enzymes such as NADases. Brain homogenates are disrupted and clarified by sonication (0.5 second per cycle, 45 cycles, 50% power) followed by centrifugation at 12,000 rpm, RT for 5 minutes. Supernatants are collected and fractionated using Amicon Ultra-2 centrifugal units with a 30 kDa cut-off. Flow-through is collected and NAD is measured using a cycling assay (NAD+/NADH-Glo™ (Promega). The NAD+/NADH-Glo™ assay is a bioluminescent, homogeneous assay for the detection of total NAD metabolite levels comprising the oxidized and reduced forms of NAD (NAD+ and NADH, respectively). An NAD cycling enzyme is used to convert NAD+ to NADH. In the presence of NADH, the provided reductase enzyme reduces a proluciferin reductase substrate to form luciferin. Luciferin is then quantified using Ultra-Glo recombinant luciferase, and the light signal produced is proportional to the amount of NAD+ and NADH in the sample. Cycling between NAD+ and NADH by the NAD cycling enzyme and reductase increases assay sensitivity and provides selectivity for nonphosphorylated NAD+ and NADH compared to the phosphorylated forms NADP+ and NADPH. Reading is performed between 10 and 50 minutes during the linear increase of the signal in a luminescence reader. NAD values are normalized by total protein concentrations.

FIG. 1C shows that mouse blood was collected in 1 ml collection microtubes coated with K3EDTA to prevent clotting. Whole blood was diluted 1/120 in ice-cold PBS in the presence of 1 mM NAM, prior to processing similarly to brain homogenates.

FIG. 1D shows that mouse CSF was collected in 50011.1 collection microtubes, centrifuged for 5 minutes at 1500 g, and the supernatant was collected and frozen in dry ice. CSF was diluted 1/20 in ice-cold PBS in the presence of 1 mM NAM, prior to processing similarly to brain homogenates.

FIG. 1E shows that human CSF from a healthy control was diluted 1/2 in ice-cold PBS in the presence of 1 mM NAM, prior to processing similarly to brain homogenates. FIGS. 1A, 1B, 1C, 1D, 1E: Linear regression was performed in Microsoft Excel v16.

FIG. 1F shows that NAD protection from degradation by NADases. Two different inhibitors, NAM or apigenin (an inhibitor of the NADase CD38[36]) were added or not at the concentrations indicated into different aliquots of a brain sample after homogeneisation. Error bars of duplicate samples are shown. NAM provided potent protection as opposed to the absence of protection conferred by apigenin.

FIG. 1G shows that optimization of NAD protection by NAM. NAM was added at various concentrations in different aliquots of a brain sample after homogeneisation. Error bars of duplicate samples are shown. NAM at 1 mM provided the most potent NAD protection.

FIG. 1H shows that signal decrease after 20 minutes in a brain sample if the fractionation phase is omitted. The brain sample was processed as described in FIG. 1B except that the clarified supernatant was not fractionated prior to NAD measurement.

FIGS. 1I and 1J show that optimization of the dilution factor for blood NAD measurement to avoid signal saturation. NAD was measured in blood samples from 9 individual mice in two consecutive experiments. In the first experiment the blood sample was diluted 60× leading to signal saturation after 30 minutes of reading time (FIG. 1I). The blood sample was then further diluted to achieve a 120× dilution, and the readings were linear druing the entire reading window up to 70 minutes (FIG. 1J). Except for the dilution factors, the blood sample used for this experiment was processed as described in FIG. 1C.

FIGS. 2A and 2B are two graphs showing a progressive decline in NAD levels correlating with disease stage in the brains of Tg(SOD1*G93A) mice.

FIG. 2A shows that brain NAD levels were measured in whole brain homogenates of sick ALS mice at ALS mouse clinical scale (ALSMCS) 4 (hindlimb paresis) and compared with wild-type, age matched congenic animals. ALS mice (n=9); control mice (n=6). Sick ALS mice were euthanized at 144±2 days of age. ALS vs control: p=0.03 in the unpaired t-test with Welch's correction.

FIG. 2B shows that brain NAD levels were measured in whole brain homogenates of sick ALS mice at ALSMCS 5 (clinical end point with hindlimb paralysis) and compared with wild-type, age matched congenic animals. ALS mice (n=8); control mice (n=8). Sick ALS mice were euthanized at 152±2 days of age. ALS vs control: p=0.0005 in the unpaired t-test with Welch's correction. FIGS. 2A-2B: median values are shown.

FIGS. 3A and 3B are graphs showing the neuroprotective and NAD-restoring dose-response activity curves of SR1457 (FIG. 3A) and its analogue SR005 (FIG. 3B) in the viability and NAD assays. Cell viability in the presence of toxic prion protein (TPrP), red; NAD quantification in the presence of TPrP, black; cell viability in naive cells, green. The assays were performed in 1536-well plates. Neuroblastoma cells were treated with 4 μg/ml TPrP for 3 days in 5 μl volume total. TPrP was prepared as described in ref.[37]. Compounds were added in a 10-point, 4 logs titration at the doses indicated. Cell viability was measured using CELLTITER-GLO® (Promega). NAD was quantified using NAD+/NADH-GLO™ (Promega).

FIGS. 4A and 4B are graphs showing the dose-dependent nicotinamide phosphoribosyl transferase (NAMPT) activation by SR1457. A colorimetric NAMPT activity assay was used (Abcam). The assay was performed according to the manufacturer's instructions except that human NAMPT was replaced by mouse NAMPT (Fisher Scientific). The activity of SR1457 was tested at the doses indicated (04). Single data points are shown. Enzymatic activity rate was calculated by the formula: (A50-A20)/(T50-T20) where A is the OD450 at each time point T (min).

FIG. 5 is a graph showing that oral treatment with SR005 delays loss of muscle strength in the Tg(SOD1*G93A) mouse model of ALS. Mice (all female) received 6 mg/kg SR005 daily in their drinking water from 47 days of age. Sugar-free strawberry flavored gelatin (Jell-O®) is used for taste-masking and 4% DMSO for compound solubilization. Vehicle controls received the same mixture without compound. Hanging-wire tests were performed as described^{5,6}. Average±SEM are shown. Statistical analysis was performed with 2-way Anova; n=12; P<0.001 for all but the 145 days time point.

FIG. 6 is a graph showing the increased NAD levels in brains of SR005 treated ALS mice compared with vehicle control ALS mice. Brains were from terminally sick mice (SR005 group, n=8; vehicle group, n=13). NAD was quantified as described in FIG. 1B. Vehicle vs treated: p=0.07 in the unpaired t-test with Welch's correction.

FIGS. 7A and 7B are graphs showing the quantification of NAD in the CSF and blood of sick Tg(SOD1*G93A) ALS mice compared with wild-type, age-matched congenic mice.

FIG. 7A shows that sick ALS mice (ALSMCS 5) were euthanized at 152±2 days of age and CSF drawn. ALS vs control: p=0.03 in the unpaired t-test with Welch's correction. ALS mice (n=8); control mice (n=8).

FIG. 7B shows that sick ALS mice (ALSMCS 4) were euthanized at 144±2 days of age and cardiac blood drawn. ALS mice (n=9); control mice (n=8). ALS vs control: p=0.01 in the unpaired t-test with Welch's correction. FIGS. 7A-7B: median values are shown.

FIG. 8 is a series of graphs showing the pharmacological modulation of blood NAD levels by SR005 or NAM administration. Three C57BL/6 mice were treated orally with either 6 mg/kg SR005 or 100 mg/kg NAM and 20-30 μl blood drawn from the facial vein at the time points indicated for a longitudinal follow-up of whole blood NAD levels in each individual mouse. Blood was collected and NAD detected as described in FIGS. 1A-1C. NAD values were normalized by baseline level for each mouse. Each line represents data for one mouse. Repeated measures ANOVA, where Geisser-Greenhouse sphericity correction was used, indicated that NAD levels changed over time (F(1.2,2.3)=39, p=0.017).

### DETAILED DESCRIPTION

The present disclosure is based on the discovery of an easy-to-implement, rapid and analytically robust method for the detection of NAD in brain tissue, body fluids and other biological samples of rodents and humans. Based on the method, it was discovered that there is a reduction of NAD levels in the brains of ALS mice, and restoration of brain NAD levels in mice correlating with clinical efficacy of a neuroprotective compound. It was also discovered that there is an increase of NAD levels in the CSF and blood of ALS mice providing a means to diagnose the disease, monitor its progression and monitor the effects of a disease-modifying treatment in body fluids. Finally, it was also shown that the pharmacodynamic effect of an NAD elevating drug can be monitored by quantification of NAD in blood.

NAD is a metabolite serving in various types of biological reactions regulating energy production, protein processing, calcium signaling, DNA repair and transcription[38]. NAD levels are therefore tightly regulated at a cellular, body fluid and tissue/organ level. NAD metabolism is impaired as a result of exposure to misfolded prion protein[39]. Such impairment has also been shown in experimental models of PD and ALS[40, 41]. NAD dysregulation is now also recognized as being involved in AD[42, 43], aging[44, 45], neuronal degeneration associated with multiple sclerosis[46], hearing loss[47], retinal damage[48], traumatic brain injury[49], and axonopathy[50]. Substantial decreases in NAD levels are found in degenerative renal conditions[51]. NAD augmentation such as NAD administration or increased NAD synthesis by enzyme overexpression has been shown to mitigate brain ischemia [52], cardiac ischemia/reperfusion injury[53, 54] and acute kidney injury[51]. NAD metabolism has also been shown to be altered in murine models of type 2 diabetes (T2D)[55, 56].

Accordingly, NAD measurement in body fluids or tissues has utility in diagnosing ALS and other neurodegenerative, degenerative and metabolic conditions where NAD metabolism is compromised. Since we showed that NAD levels are a molecular signature of cellular demise that occurs progressively in these diseases, with both a molecular signature in brain tissue and a distinct molecular signature in CSF and blood, it is proposed herein, that NAD quantification in biological samples is utilized as a prognostic biomarker and a biomarker for disease progression. It was also demonstrated herein that NAD levels can be pharmacologically modulated in blood and the CNS, therefore blood and/or CSF and/or other biological sample NAD measurement can be used as a pharmacodynamic biomarker to evaluate the disease-modifying effect of an investigational drug candidate in clinical trials and as an outcome measure of treatment efficacy. Further, biofluid NAD levels could be ordered to be measured in a routine laboratory and monitored by a physician in the clinical practice to monitor patient response to an NAD-elevating or other disease-modifying treatment. Examples of neuroprotective drugs can be found at Drugbank, Accession Number DBCAT000653 listed in the below Table 1. In addition, compounds SR1457 and SR005 described herein have been shown to be protective in murine models of ALS and PD (described in patent application PCT/US20/32903).

TABLE 1

| DRUG | DRUG DESCRIPTION |
|---|---|
| Topiramate | An anticonvulsant drug used in the |
| | control of epilepsy and in the |
| | prophylaxis and treatment of migraines. |
| Riluzole | A glutamate antagonist used to treat |
| | amyotrophic lateral sclerosis. |
| Methylprednisolone | A corticosteroid used to treat |
| | inflammation or immune reactions |
| | across a variety of organ systems, |
| | endocrine conditions, and neoplastic |
| | diseases. |
| Rivastigmine | A cholinesterase inhibitor used to |
| | treat mild to moderate dementia in |
| | Alzheimer's and Parkinson's. |
| Selegiline | A monoamine oxidase inhibitor used |
| | to treat major depressive disorder |
| | and Parkinson's. |
| Cilostazol | An antiplatelet agent and vasodilator |
| | used for the symptomatic relief of |
| | intermittent claudication. |
| Rasagiline | An irreversible inhibitor of monoamine |
| | oxidase used for the symptomatic |
| | management of idiopathic Parkinson's |
| | disease as initial monotherapy and as |
| | adjunct therapy to levodopa. |
| Tenocyclidine | Because of its high affinity for |
| | the phencyclidine binding site on |
| | the NMDA receptor, the 3H radiolabelled |
| | form of tenocyclidine is widely used |
| | in research into NMDA receptors. |
| 7-Nitroindazole | Not Available |
| N-(3- | Not Available |
| Propylcarbamoyloxirane- | |
| 2-Carbonyl)-Isoleucyl- | |
| Proline | |
| Huperzine A | Investigated for use/treatment in |
| | alzheimer's disease. |
| SGS-742 | Investigated for use/treatment in |
| | alzheimer's disease, attention deficit/ |
| | hyperactivity disorder (ADHD), memory |
| | loss, and schizophrenia and |
| | schizoaffective disorders. |
| D-JNKI-1 | Investigated for use/treatment in |
| | hearing loss. |
| Nalmefene | An opioid antagonist used in conjunction |
| | with psychosocial support to help adults |
| | with alcohol dependence reduce alcohol |
| | consumption. |
| Ziconotide | An N-type calcium channel antagonist |
| | used to manage patients with severe |
| | chronic pain who cannot tolerate, or |
| | who have not responded adequately to |
| | other treatments such as intrathecal |
| | morphine and systemic analgesics. |
| Dexanabinol | Investigated for use/treatment in |
| | traumatic brain injuries and neurologic |
| | disorders. |
| Remacemide | Investigated for use/treatment in |
| | epilepsy, huntington's disease, and |
| | parkinson's disease. |
| Clomethiazole | Investigated for use/treatment in |
| | strokes. |
| Propentofylline | Investigated for use/treatment in |
| | alzheimer's disease. |
| Z-Val-Ala-Asp | Non-methylated, competitive, and |
| fluoromethyl | irreversible inhibitor of caspase 1, |
| keton | as well as other caspases, 1 which |
| | can be used directly with purified |
| | enzymes. |
| Piracetam | Indicated in adult patients suffering |
| | from myoclonus of cortical origin, |
| | irrespective of aetiology, and should |
| | be used in combination with other |
| | anti-myoclonic therapies. |
| Epigallocatechin | Epigallocatechin gallate has been |
| gallate | investigated for the treatment of |
| | Hypertension and Diabetic Nephropathy. |
| Vinpocetine | Vinpocetine has been investigated for |
| | the treatment of Epilepsy. |
| Tempol | Tempol has been used in trials studying |
| | the treatment of Anal Cancer. |
| Butylphthalide | Butylphthalide has been used in trials |
| | studying the prevention of Restenosis. |
| Eliprodil | Eliprodil has been used in trials |
| | studying the treatment of Parkinson |
| | Disease and Movement Disorders. |
| Tirilazad | Tirilazad has been used in trials |
| | studying the treatment of Spinal |
| | Cord Injury. |
| Nefiracetam | Nefiracetam has been used in trials |
| | studying the treatment of Alzheimer's |
| | Disease. |
| Gacyclidine | Gacyclidine has been used in trials |
| | studying the treatment of Tinnitus. |
| Nizofenone | Not Annotated |
| Meclofenoxate | Not Annotated |
| Linopirdine | Not Annotated |
| Fosfructose | Not Annotated |
| Methylprednisolone | A water soluble corticosteroid used |
| hemisuccinate | to treat severe allergic reactions, |
| | dermatologic diseases, endocrine |
| | disorders, gastrointestinal diseases, |
| | hematological disorders, neoplastic |
| | diseases, nervous system conditions, |
| | ophthalmic diseases, renal diseases, |
| | respiratory diseases, and rheumatic |
| | disorders. |
| Dextrorphan | Not Annotated |
| Ebselen | Ebselen has been investigated for the |
| | treatment and basic science of Meniere's |
| | Disease, Type 2 Diabetes Mellitus, and |
| | Type 1 Diabetes Mellitus. |
| Almitrine | For the treatment of chronic obstructive |
| | pulmonary disease. |
| Brimapitide | Brimapitide is under investigation in |
| | clinical trial NCT01570205 (Safety, |
| | Tolerability and PK of a Single iv |
| | Infusion of 10, 40, and 80 μg/kg |
| | XG-102 Administered to Healthy |
| | Volunteers). |
| Edaravone | A free radical scavenger used to |
| | delay the progression of ALS. |

The present disclosure has utility as a biomarker supporting the development of treatments for neurodegenerative diseases such as Alzheimer's and Parkinson's diseases (AD, PD), prion diseases and amyotrophic lateral sclerosis (ALS). These diseases share a common pathogenic mechanism, i.e. the misfolding and aggregation of endogenous proteins that become toxic for neurons. PD and ALS are heterogeneous diseases from a clinical, genetic and molecular standpoint.

The Alzheimer's, Parkinson's and ALS fields have suffered from slow and inefficient clinical trials due to heterogeneous patients populations and the use of clinical rating scales as main, if not sole, outcome measure. CSF and/or blood and/or other biosample NAD measurements may provide a means for patient stratification and an objective quantifiable measure of treatment efficacy, thereby accelerating the demonstration that a drug candidate has utility. The FDA has instituted accelerated approval regulations whereby a fast-tracked drug candidate may be approved based on a surrogate biomarker or an intermediate clinical endpoint. It is envisaged herein, that the use of body fluid(s) NAD levels be a surrogate biomarker for neurodegenerative diseases. It is further envisaged that blood or other body fluid NAD measurements will have utility in the clinical practice to determine patient eligibility for a neuroprotective or NAD elevating treatment, verify patient treatment compliance and response to treatment.

Metabolic disorders induced by obesity include such obesity-related metabolic diseases that insulin resistance (a state in which insulin does not work well) is strongly involved in the development, such as diabetes mellitus and fatty liver. The hormone that lowers blood glucose in vivo is insulin, which is synthesized and secreted in the pancreatic .beta. cell. A liver is responsible for approximately 90% of gluconeogenesis, and insulin in normal condition suppresses gluconeogenesis in the liver to maintain the constant blood glucose, and also controls fat synthesis in the liver to be constant. During obesity progression, in contrast, most of the cases possess hyperinsulinemia simultaneously with hyperglycemia, not only the development of diabetes mellitus and fatty liver, but also the development of hypertension and hyperlipidemia is promoted. Moreover, hyperinsulinemia is also involved in the development of cardiocerebrovascular lesions. In addition, insulin resistance is found in fat and muscle, and involved in the development of obesity-related metabolic disease. In contrast, hepatic insulin resistance occurs at an early period in a manner independent of insulin resistance in other organ.

Insulin resistance in obesity is separately observed between gluconeogenesis system and lipid synthesis system, and selective insulin resistance occurs in a liver (selective hepatic insulin resistance). Consequently, the gluconeogenesis system becomes insulin-resistant, causing hyperglycemia is prevented from being suppressed in spite of hyperinsulinemia. Furthermore, when insulin resistance is preserved for a long period of time, pancreatic .beta. cells become disable to maintain high-insulin condition, and the β-cell function gradually becomes exhausted to result in a decrease in insulin secretion, and finally diabetes mellitus is developed.

In contrast, the lipid synthesis system is not vulnerable to insulin-resistant, and hyperinsulinemia promotes lipid synthesis in the liver to lead to the development of non-alcoholic fatty liver disease (NAFLD), which is characterized by excessive accumulation of triglyceride (Tg), and the development of NAFLD further worsen the insulin resistance. Most of the hepatic lipid in NAFLD is derived from enhancement of the lipid synthesis system in the liver, and fatty acid oxidation is reduced in the liver with NAFLD. Therefore, excessive Tg synthesized is not metabolized and Tg is accumulated together with cholesterol in lipid droplet of hepatocyte. Moreover, hyperinsulinemia condition over a long period of time promotes fibrosis and inflammation of the liver with NAFLD together with obesity-related metabolic diseases such as hyperglycemia, hyperlipidemia, and hypertension. Excessively accumulated Tg and cholesterol in the liver act as lipotoxicity to injure hepatocyte, resulting in the development of non-alcoholic steatohepatitis (NASH). Among NAFLD cases untreated for 10 years or longer, 20 to 27% progress into NASH, and hepatic cirrhosis, hepatic failure and liver cancer develop from NASH condition with very high probability.

Other examples of metabolic diseases or disorders include lysosomal storage disorders. Various enzyme deficiencies inside lysosomes can result in buildup of toxic substances, causing metabolic disorders including: Hurler syndrome (abnormal bone structure and developmental delay), Niemann-Pick disease (babies develop liver enlargement, difficulty feeding, and nerve damage), Tay-Sachs disease (progressive weakness in a months-old child, progressing to severe nerve damage; the child usually lives only until age 4 or 5), Gaucher disease (bone pain, enlarged liver, and low platelet counts, often mild, in children or adults), Fabry disease (pain in the extremities in childhood, with kidney and heart disease and strokes in adulthood; only males are affected), Krabbe disease (progressive nerve damage, developmental delay in young children; occasionally adults are affected).

Other examples of metabolic diseases include: Galactosemia, Maple syrup urine disease, Phenylketonuria (PKU), Glycogen storage diseases, Mitochondrial disorders, Friedreich ataxia, Peroxisomal disorders (e.g. Zellweger syndrome, Adrenoleukodystrophy), Metal metabolism disorders, Wilson disease, Hemochromatosis, Organic acidemias, Urea cycle disorders.

Pharmaceutical Agents

Any composition described herein can be administered to any part of the host's body for subsequent delivery to a target cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 50-, 100-, 150-, or more fold). Encapsulation of the compounds in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

The compositions described herein are suitable for use in a variety of drug delivery systems. Additionally, in order to enhance the in vivo serum half-life of the administered compound, the compositions may be encapsulated, introduced into the lumen of liposomes, prepared as a colloid, or other conventional techniques may be employed which provide an extended serum half-life of the compositions. A variety of methods are available for preparing liposomes, as described in, e.g., Szoka, et al., U.S. Pat. Nos. 4,235,871, 4,501,728 and 4,837,028 each of which is incorporated herein by reference. Furthermore, one may administer the drug in a targeted drug delivery system, for example, in a liposome coated with a tissue specific antibody. The liposomes will be targeted to and taken up selectively by the organ.

The appropriate dose of the compound is that amount effective to prevent occurrence of the symptoms of the disorder or to treat some symptoms of the disorder from which the patient suffers. By “effective amount”, “therapeutic amount” or “effective dose” is meant that amount sufficient to elicit the desired pharmacological or therapeutic effects, thus resulting in effective prevention or treatment of the disorder. Preferably, the effective amount is sufficient to obtain the desired result, but insufficient to cause appreciable side effects.

Dosage, toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compositions lies preferably within a range of circulating concentrations that include the ED₅₀ with little or no toxicity. The dosage may vary within this range depending upon the dosage form employed and the route of administration utilized. For any composition used in the method of the disclosure, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As described, a therapeutically effective amount of a composition (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the disclosure can include a single treatment or a series of treatments.

The effective dose can vary, depending upon factors such as the condition of the patient, the severity of the viral infection, and the manner in which the pharmaceutical composition is administered. The effective dose of compounds will of course differ from patient to patient, but in general includes amounts starting where desired therapeutic effects occur but below the amount where significant side effects are observed. For human patients, the effective dose of typical compounds generally requires administering the compound in an amount of at least about 1, often at least about 10, and frequently at least about 25 μg/24 hr/patient.

A subject is effectively treated whenever a clinically beneficial result ensues. This may mean, for example, a complete resolution of the symptoms of a disease, a decrease in the severity of the symptoms of the disease, or a slowing of the disease's progression. These methods can further include the steps of a) identifying a subject (e.g., a patient and, more specifically, a human patient) who has a certain disease to be treated; and b) providing to the subject the compositions comprising at least one therapeutic and/or a secondary active agent embodied herein.

The methods disclosed herein can be applied to a wide range of species, e.g., humans, non-human primates (e.g., monkeys), horses or other livestock, dogs, cats, ferrets or other mammals kept as pets, rats, mice, or other laboratory animals.

The duration of treatment with any composition provided herein can be any length of time from as short as one day to as long as the life span of the host (e.g., many years). For example, a compound can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compounds can be administered once (or twice, three times, etc.) daily, weekly, monthly, or yearly.

The dosage required will depend on the route of administration, the nature of the formulation, the nature of the patient's illness, the patient's size, weight, surface area, age, and sex, other drugs being administered, and the judgment of the attending clinicians. Wide variations in the needed dosage are to be expected in view of the variety of cellular targets and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 50-, 100-, 150-, or more fold). Encapsulation of the compounds in a suitable delivery vehicle (e.g., polymeric microparticles or implantable devices) may increase the efficiency of delivery.

Any method known to those in the art can be used to determine if a particular response is induced. Clinical methods that can assess the degree of a particular disease state can be used to determine if a response is induced. The particular methods used to evaluate a response will depend upon the nature of the patient's disorder, the patient's age, and sex, other drugs being administered, and the judgment of the attending clinician.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN’, PLURONICS’ or polyethylene glycol (PEG). Pharmaceutically acceptable excipients are further described herein.

In one example, one or more of the active agents may be formulated into liquid pharmaceutical compositions, which are sterile solutions, or suspensions that can be administered by, for example, intravenous, intramuscular, subcutaneous, or intraperitoneal injection. Suitable diluents or solvent for manufacturing sterile injectable solution or suspension include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

In some examples, the pharmaceutical composition described herein comprises liposomes containing one of the active agent which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The active agents may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

The pharmaceutical compositions to be used for in vivo administration must be sterile. This is readily accomplished by, for example, filtration through sterile filtration membranes. Therapeutic compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g. Tween™ 20, 40, 60, 80 or 85) and other sorbitans (e.g. Span™ 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™ Liposyn™, Infonutrol™, Lipofundin™ and Lipiphysan™. The active ingredient may be either dissolved in a pre-mixed emulsion composition or alternatively it may be dissolved in an oil (e.g. soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing a therapeutic and/or diagnostic agent with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Film-Forming Polymers for Coating Capsules: The film-forming composition can be used to prepare soft or hard shell gelatin capsules which can encapsulate a liquid or semi-solid fill material or a solid tablet (e.g., SOFTLET™) containing an active agent and one or more pharmaceutically acceptable excipients. Alternatively, the composition can be administered as a liquid with an active agent dissolved or dispersed in the composition. Exemplary film-forming natural polymers include, but are not limited to, gelatin and gelatin-like polymers. In certain embodiments, the film-forming natural polymer is gelatin. A number of other gelatin-like polymers are available commercially. The film-forming natural polymer is present in an amount from about 20 to about 40% by weight of the composition, or from about 25 to about 40% by weight of the composition.

The film-forming composition can be used to prepare soft or hard capsules using techniques well known in the art. For example, soft capsules are typically produced using a rotary die encapsulation process. Fill formulations are fed into the encapsulation machine by gravity. The capsule shell can contain one or more plasticizers selected from the group consisting of glycerin, sorbitol, sorbitans, maltitol, glycerol, polyethylene glycol, polyalcohols with 3 to 6 carbon atoms, citric acid, citric acid esters, triethyl citrate and combinations thereof. In addition to the plasticizer(s), the capsule shell can include other suitable shell additives such as opacifiers, colorants, humectants, preservatives, flavorings, and buffering salts and acids.

Opacifiers are used to opacify the capsule shell when the encapsulated active agents are light sensitive. Suitable opacifiers include titanium dioxide, zinc oxide, calcium carbonate, and combinations thereof. Colorants can be used for marketing and product identification/differentiation purposes. Suitable colorants include synthetic and natural dyes and combinations thereof.

Humectants can be used to suppress the water activity of the soft gel. Suitable humectants include glycerin and sorbitol, which are often components of the plasticizer composition. Due to the low water activity of dried, properly stored soft gels, the greatest risk from microorganisms comes from molds and yeasts. For this reason, preservatives can be incorporated into the capsule shell. Suitable preservatives include alkyl esters of p-hydroxy benzoic acid such as methyl, ethyl, propyl, butyl and heptyl (collectively known as “parabens”) or combinations thereof.

Ultrasound delivery: Ultrasound delivery is based on the concept of noninvasive delivery of focused ultrasound pulses that generally comprise a lipid or polymer shell, a stabilized gas core, and a diameter of less than 10 mm. In other words, the acoustic energy, such as ultrasound, can be directed by simple aiming techniques, such as physically orienting one or more transducers on a headpiece, thereby eliminating the complexities of electronic focusing and reduces the need for image guidance. This treatment also has the advantage of treating conditions where the precise site of therapy is not well defined. A highly focused approach is more likely to be unsuccessful or only partially cover the targeted region.

Acoustic energy, such as ultrasound, can be applied to the entire brain or a region of the brain. A region of the brain may be a hemisphere or forebrain. The region may be at least 25% by volume of the brain. The region of the brain may be one that is known to be associated with pathogenic protein deposition such as amyloid beta (Aβ). The particular regions of the brain to be targeted for effective treatment will differ depending on the disease. For example, for Alzheimer's disease the areas that may be targeted include the hippocampus, temporal lobe and/or basal forebrain, more specifically, the hippocampus, mamillary body and dentate gyrus, posterior cingulate gyms, and temporal lobe. For Frontotemporal Dementia the brain region to be targeted includes the cortex. For Amyotrophic Lateral Sclerosis the region to be targeted includes the spinal cord, motor cortex, brain stem.

Identifying a region of the brain to which acoustic energy is applied may include determining a volume of the brain on the basis of symptoms displayed by the subject, typically clinically observable or biochemically detectable symptoms, or determining a volume of the brain on the basis of a known association with a neurodegenerative disease, in particular those associated with protein oligomers, aggregates or deposits, or determining a volume of the brain including a volume surrounding an site having extracellular protein in a pathogenic form, such as oligomers, an aggregate or deposit.

The focus of the acoustic energy source, typically an scanning ultrasound transducer, may be moved in a pattern with space between the subject sites of application over a region of the brain as described herein or the entire brain. The focus may be moved by a motorised positioning system. In a preferred form, the methods of the invention involve the application of focussed ultrasound to a plurality of locations in the brain. The focussed ultrasound may be applied at 2, 3, 4, 5, 6, 7, 8, 9, 10 or more locations in the brain or on each hemisphere.

It is also contemplated that any disease, condition or syndrome that is a consequence of or associated with a neurodegenerative, degenerative or metabolic disease, e.g. aggregation or deposition of tau proteins in the brain, may be treated by a method of the invention. In addition, a symptom of a disease, condition or syndrome that is a consequence of or associated with neurodegenerative, degenerative or metabolic disease, may be reduced in severity or incidence by a method of the invention.

Increasing the permeability of the blood-brain barrier can be promoted by various agents. These agents are based on the principle that biologically inert and preformed microbubbles, with either a lipid or polymer shell, a stabilized gas core, and a diameter of less than 10 μm, can be systemically administered and subsequently exposed to noninvasively delivered focused ultrasound pulses.

Scanning ultrasound may be combined with microbubbles to disrupt the blood-brain barrier (BBB) which is achieved by mechanical interactions between the microbubbles and the blood vessel wall as pulsed focused ultrasound is applied, resulting in cycles of compression and rarefaction of the microbubbles. This leads to a transient disruption of tight junctions and the uptake of blood-borne factors by the brain. Microbubbles within the target volume become “acoustically activated” by what is known as acoustic cavitation. In this process, the microbubbles expand and contract with acoustic pressure rarefaction and compression over several cycles. This activity has been associated with a range of effects, including the displacement of the vessel wall through dilation and contraction. More specifically, the mechanical interaction between ultrasound, microbubbles, and the vasculature transiently opens tight junctions and facilitates transport across the BBB.

The microbubble agent can be any agent known in the art including lipid-type microspheres or protein-type microspheres or a combination thereof in an injectable suspension. For example, the agent can be selected from the group consisting of Octafluoropropane/Albumin (Optison), a perflutren lipid micro sphere (Definity), Galactose-Palmitic Acid microbubble suspension (Levovist) Air/Albumin (Albunex and Quantison), Air/Palm itic acid (Levovist/SHU508A), Perfluoropropane/Phospholipids (MRX115, DMP115), Dodecafluoropentane/Surfactant (Echogen/QW3600), Perfluorobutane/Albumin (Perfluorocarbon exposed sonicated dextrose albumin), Perfluorocarbon/Surfactant (QW7437), Perfluorohexane/Surfactant (Imagent/AF0150), Sulphur hexafluoride/Phospholipids (Sonovue/BR1), Perfluorobutane/Phospholipids (BR14), Air/Cyanoacrylate (Sonavist/SHU563A), and Perfluorocarbon/Surfactant (Sonazoid/NC100100).

The microbubble agent may be provided as a continuous infusion or as a single bolus dose. A continuous infusion of microbubble, preferably provided over the duration of the acoustic energy application, would be preferred. Typically, the microbubble agent is delivered intravenously through the systemic circulation. For methods of the invention that include the use of an agent such as a microbubble or other cavitation based promotion of blood-brain barrier permeability, the agent may be localized at, or near, or in a region that is targeted with the ultrasound such that the potential of unwanted damage from cavitation effects is minimised.

The applying step, for the delivery of acoustic energy, may comprise the delivery of acoustic energy from an acoustic energy source through a fluid coupler applied directly to the head of the subject. In this application, the fluid coupler may be applied to only one side or aspect of the subject's head. The head may be an unmodified head or a head with a surgically created window in the skull-the fluid coupler being in contact with the window. The acoustic energy may be generated by an unfocused acoustic energy transducer or a phased array acoustic energy transducer (i.e., focused acoustic energy). Significantly, the phased array acoustic energy transducer may be a diagnostic phased array. Diagnostic phased arrays are generally of lower power and are commonly available. The fluid coupler may comprise a contained volume of fluid (e.g., about 50 cc, about 100 cc, about 200 cc, about 400 cc, about 500 cc, about 600 cc or about 1 litre). The fluid may be, for example, water, acoustic energy gel, or a substance of comparable acoustic impedance. The fluid may be contained in a fluid cylinder with at least a flexible end portion that conforms to the subject's head. In other embodiments, the contained volume of fluid may be a flexible or elastic fluid container.

Increased permeability of the blood-brain barrier may be determined by any suitable imaging method. Preferably, the imaging method is MRI, an optical imaging method, positron emission tomography (PET), computerized tomography (CT) or computerized axial tomography (CAT) or ultrasound. If a level of acoustic energy is applied, the increased permeability of the blood-brain barrier could then be determined by any one of the methods described herein and an increased level of acoustic energy could be subsequently applied until the permeability of the bloodbrain barrier had increased to a clinically relevant level. The permeability of the BBB may also be determined by a number of known techniques including injection with Evans blue dye that binds to albumin, a protein that is normally excluded from the brain.

Any ultrasound parameters that result in clinically safe application of acoustic energy are useful in the invention. Typically, the ultrasound parameters that are preferred as those that result in an increase the permeability of the blood-brain barrier, or activate microglia phagocytosis. Various ultrasound parameters can be manipulated to influence the permeability increase in the blood-brain barrier and these include pressure amplitude, ultrasound frequency, burst length, pulse repetition frequency, focal spot size and focal depth. Several parameters are now described that are useful in a method of the invention.

Focal spot size useful in a method of the invention includes about a 1 mm to 2 cm axial width. Typically, the focal spot size has an axial width of about 1 mm to 1.5 cm, preferably 1 mm to 1 cm, even more preferably 1 mm to 0.5 cm. The length of the focal spot may be about 1 cm to as much as about 15 cm, preferably 1 cm to 10 cm, even ore preferably 1 cm to 5 cm. The focal size useful in a method of the invention is one that allows an increase in the permeability of the blood-brain barrier of the subject.

The focal depth of the ultrasound generally depends on the areas of the brain affected by the disease. Therefore, the maximum focal depth would be the measurement from the top of the brain to the base, or about 10 to about 20 cm. Focal depth could be altered by electronic focusing, preferably by using an annular array transducer. The focal depth allows application to the cortical layer which, for example, may be up to 4 cm deep.

Typically the ultrasound is applied in continuous wave, burst mode, or pulsed ultrasound. Preferably the ultrasound is applied in burst mode, or pulsed ultrasound. Pulse length parameters that are useful in a method the invention include between about 1 to about 100 milliseconds, preferably the pulse length or burst length is about 1 to about 20 milliseconds. Exemplary burst mode repetition frequencies can be between about 0.1 to 10 Hz, 10 Hz to 100 kHz, 10 Hz to 1 kHz, 10 Hz to 500 Hz or 10 Hz to 100 Hz.

The duty cycle (% time the ultrasound is applied over the time) is given by the equation duty cycle=pulse length×pulse repetition frequency.times.100. Typically, the duty cycle is from about 0.1% to about 50%, about 1% to about 20%, about 1% to about 10%, or about 1% to about 5%.

The ultrasound pressure useful in a method of the invention is the minimum required to increase the permeability of the blood-brain barrier. The human skull attenuates the pressure waves of the ultrasound which also depends on the centre frequency of the transducer, with lower centre frequencies of the ultrasound transducer causing better propagation and less attenuation. A non-limiting example of ultrasound pressure is between MPa to 3 MPa, preferably about 0.4 or 0.5 MPa. Typically this pressure is applied to the skull, i.e. transcranially. The mechanical index characterises the relationship between peak negative pressure amplitude in situ and centre frequency with mechanical index=Pressure (MPa)/sqrt centre frequency (MHz) if this mechanical index was free from attenuation/measured from within the skull, the mechanical index would be between about and about 2, preferably about 0.1 to 1 or 0.1 to 0.5.

A non-limiting example of a system that is able to open the blood-brain barrier is the TIPS system (Philips Research). It consists of a focused ultrasound transducer that generates a focused ultrasound beam with a centre frequency of 1-1.7 MHz focal depth of 80 mm, active outer diameter 80 mm, active inner diameter 33.5 mm which is driven by a programmable acoustic signal source within the console and attached to a precision motion assembly. An additional example of a system that is able to generate an ultrasound beam suitable for blood-brain barrier disruption is the ExAblate Neuro (Insightec) system. Suitable parameters for blood-brain barrier opening in humans such as centre frequency and microbubble dosage may be different to that in mice.

For any of the method or apparatus of the invention, the ultrasound transducer may have an output frequency of between 0.1 to 10 MHz, or 0.1 to 2 MHz. The ultrasound may be applied for a time between 10 milliseconds to 10 minutes. The ultrasound may be applied continuously or in a burst mode.

Image contrast agents, used in any methods of the invention, may be selected from the group consisting of magnetic resonance contrast agents, x-ray contrast agents (and x-ray computed tomography), optical contrast agents, positron emission tomography (PET) contrast agents, single photon emission computer tomography (SPECT) contrast agents, or molecular imaging agents. For example, the imaging contrast agent may be selected from the group consisting of gadopentetate dimeglumine, Gadodiamide, Gadoteridol, gadobenate dimeglumine, gadoversetamide, iopromide, lopam idol, Ioversol, or Iodixanol, and lobitridol.

The frequency of application of the ultrasound would generally depend on patient severity. The parameters of the ultrasound and the treatment repetition are such that there is an increase in permeability of the blood-brain barrier but preferably wherein there is no, or clinically acceptable levels of, damage to parenchymal cells such as endothelial or neuronal damage, red blood cell extravasation, haemorrhage, heating and/or brain swelling. Any method of the invention may further include performing magnetic resonance imaging on a subject comprising the steps of (a) administering a magnetic resonance contrast agent to a subject through the blood-brain barrier using any of the methods of the invention and performing magnetic resonance imaging on said subject. In this context the use of magnetic resonance imaging is to confirm the increase in permeability of the blood-brain barrier and not to locate the presence of a pathogenic protein.

Another embodiment of the invention involves providing an imaging contrast agent to the whole brain including the steps of administering an imaging contrast agent into the bloodstream of said subject; and applying ultrasound to the brain of said subject to open the bloodbrain barrier to allow the imaging contrast agent to cross the blood-brain barrier. The imaging contrast agent can be administered to the subject simultaneously or sequentially with the application of the ultrasound. In this embodiment the sequential administration of the contrast agent can be prior to or post application of the ultrasound such as SUS. In a preferred embodiment, any of the agents described herein may be administered to the bloodstream between 0 to 4 hours, between 2 to 4 hours or between 3-4 hours after ultrasound treatment using one of the methods of the invention. Preferably, the agents described herein are co-delivered.

Candidate/Test Agents

Various candidate agents can be employed in the screening methods of the invention, including any naturally existing or artificially generated agents. They can be of any chemistry class, such as antibodies, proteins, peptides, small organic compounds, saccharides, fatty acids, steroids, purines, pyrimidines, nucleic acids, and various structural analogs or combinations thereof. In some embodiments, the screening methods utilize combinatorial libraries of candidate agents. Combinatorial libraries can be produced for many types of compounds that can be synthesized in a step-by-step fashion. Such compounds include polypeptides, beta-turn mimetics, nucleic acids, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated herein by reference for all purposes). Peptide libraries can also be generated by phage display methods, See, e.g., Devlin, WO 91/18980.

In certain embodiments, a method of identifying candidate neuroprotective or disease-modifying therapeutic agents comprising contacting a cell or biological sample with a candidate agent, measuring the nicotinamide di nucleotide (NAD) levels in the cell or biological samples as compared to controls and determining whether NAD is modulated in response to the agent. In certain embodiments, an increase in NAD levels as compared to a control is indicative of a neuroprotective and/or disease-modifying therapeutic agent. In certain embodiments, the cells comprise cells obtained from a subject diagnosed with a neurodegenerative, degenerative or metabolic disorder, cell lines, transfected cells, mesenchymal cells, induced pluripotent cells (iPSCs), stem cells or combinations thereof. In certain embodiments, the assay is a high-throughput screening assay.

Candidate agents include numerous chemical classes, though typically they are organic compounds including small organic compounds, nucleic acids including oligonucleotides, peptides or antibodies. Small organic compounds suitably may have e.g. a molecular weight of more than about 40 or 50 yet less than about 2,500. Candidate agents may comprise functional chemical groups that interact with proteins and/or DNA.

Candidate agents may be obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of e.g. bacterial, fungal and animal extracts are available or readily produced.

Chemical Libraries: Developments in combinatorial chemistry allow the rapid and economical synthesis of hundreds to thousands of discrete compounds. These compounds are typically arrayed in moderate-sized libraries of small molecules designed for efficient screening. Combinatorial methods can be used to generate unbiased libraries suitable for the identification of novel compounds. In addition, smaller, less diverse libraries can be generated that are descended from a single parent compound with a previously determined biological activity.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol. Divers. 2:223-36, 1997; Fauchere et al., Peptide and nonpeptide lead discovery using robotically synthesized soluble libraries, Can J. Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, (1993)); vinylogous polypeptides (Hagihara, et al., J. Amer. Chem. Soc. 114:6568 (1992)); nonpeptidal peptidomimetics with .beta.-D-glucose scaffolding (Hirschmann, et al., J. Amer. Chem. Soc., 114:9217-9218 (1992)); analogous organic syntheses of small compound libraries (Chen, et al., J. Amer. Chem. Soc., 116:2661 (1994)); oligocarbamates (Cho, et al., Science, 261:1303 (1993)); and/or peptidyl phosphonates (Campbell, et al., J. Org. Chem. 59:658 (1994)); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Devices for the preparation of combinatorial libraries are commercially available (see, e.g., 357 MPS, 390 MPS, Advanced Chem. Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd., Moscow, RU, 3D Pharmaceuticals, Exton, Pa., Martek Bio sciences, Columbia, Md., etc.).

The screening assays of the invention suitably include and embody, animal models, cell-based systems and non-cell based systems. Identified genes, variants, fragments, or oligopeptides thereof are used for identifying agents of therapeutic interest, e.g. by screening libraries of compounds or otherwise identifying compounds of interest by any of a variety of drug screening or analysis techniques. The gene, allele, fragment, or oligopeptide thereof employed in such screening may be free in solution, affixed to a solid support, borne on a cell surface, or located intracellularly. The measurements will be conducted as described in detail in the examples section which follows.

In some embodiments, a method of identifying candidate therapeutic agents comprises screening a sample containing the specific target molecule in a high-throughput screening assay.

In another embodiment, a method of identifying therapeutic agents comprises contacting: (i) a target molecule with a candidate therapeutic agent; determining whether (i) the agent modulates a function of the peptide or interaction of the peptide with a partner molecule; or (ii) the agent modulates expression and/or function of the nucleic acid sequence of the target as measured by the light emission assays embodied herein.

In another embodiment, a method of identifying candidate therapeutic agents for treatment of disease, comprises culturing an isolated cell expressing a target molecule, administering a candidate therapeutic agent to the cultured cell; correlating the target molecules expression, activity and/or function in the presence or absence of a candidate therapeutic agent as compared to control cells, wherein a drug is identified based on desirable therapeutic outcomes. For example, a drug which modulates levels of the target molecule whereby such levels are responsible for the disease state or the target molecule modulates the activity or amount of another molecule whether upstream or downstream in a pathway. In other examples the assays measure kinase activity. In other examples, the assay measure binding partners. In other examples, the assay measures amounts of candidate therapeutic agents which provide a desired therapeutic outcome.

Another suitable method for diagnosis and candidate drug discovery includes contacting a test sample with a cell expressing a target molecule, and detecting interaction of the test agent with the target molecule, an allele or fragment thereof, or expression product of the target molecule an allele or fragment thereof.

In another preferred embodiment, a sample, such as, for example, a cell or fluid from a patient is isolated and contacted with a candidate therapeutic molecule. The genes, expression products thereof, are monitored to identify which genes or expression products are regulated by the drug.

In another aspect, the invention provides methods for diagnosing or monitoring disease progression in subjects affected, or likely to develop or having a neurodegenerative, degenerative or metabolic disease. Nicotinamide dinucleotide (NAD) can be used as a biomarker for progression of disease in a human affected by a a neurodegenerative, degenerative or metabolic disease or in an animal model of a neurodegenerative, degenerative or metabolic disease. Indeed, detecting and quantifying nicotinamide dinucleotide (NAD) levels in the brain, CNS, other tissues and body fluids will predictably reflect disease progression in a human or animal, and measuring NAD levels can be used as a biomarker to monitor the therapeutic effect of disease-modifying treatments in clinical trials. These methods entail detecting and measuring in a biological sample (e.g., a tissue or body fluid sample) from the subjects the presence and/or amounts of NAD described herein or related variants. In some methods, the biological sample is obtained from the brain of the subject.

The invention also provides engineered cells (e.g., neural cells) and transgenic animals expressing NAD or modulators thereof. The engineered cells and transgenic animal may be used in vitro or animal models to study a neurodegenerative, degenerative or metabolic disease as noted above, or to test the efficacy of therapeutic agents. The transgene is preferably present in all or substantially all of the somatic and germline cells of the transgenic animal. The polynucleotide encoding the full-length and/or mutated and/or truncated NAD or variants or modulators thereof is operably linked to one or more regulatory segments that allow the expression neuronal cells of the animal. Promoters such as the rat neuron specific enolase promoter, the prion protein promoter, human beta-actin gene promoter, human platelet derived growth factor B (PDGF-B) chain gene promoter, rat sodium channel gene promoter, mouse myelin basic protein gene promoter, human copper-zinc superoxide dismutase gene promoter, and mammalian POU-domain regulatory gene promoter can be employed in expressing the transgene. Optionally, an inducible promoter can be used. The mouse metallothionine promoter, which can be regulated by addition of heavy metals such as zinc to the mouse's water or diet, is suitable. The engineered cells or transgenic animals of the invention can be produced by the general approaches described in the art, e.g., Masliah et al., Am. J. Pathol. 148:201-10, 1996; Feany et al, Nature 404:394-8, 2000; and U.S. Pat. No. 5,811,633.

Kits

The compositions described herein can be packaged in suitable containers labeled, for example, for use as a therapy to treat a subject having a neurodegenerative, degenerative or metabolic disease. The containers can include a composition comprising at least one therapeutic agent and one or more of a suitable stabilizer, carrier molecule, flavoring, and/or the like, as appropriate for the intended use. Accordingly, packaged products (e.g., sterile containers containing one or more of the compositions described herein and packaged for storage, shipment, or sale at concentrated or ready-to-use concentrations) and kits, including at least one composition of the invention, and instructions for use, are also within the scope of the invention. A product can include a container (e.g., a vial, jar, bottle, bag, or the like) containing one or more compositions of the invention. In addition, an article of manufacture further may include, for example, packaging materials, instructions for use, syringes, delivery devices, buffers or other control reagents for treating or monitoring the condition for which prophylaxis or treatment is required.

The product may also include a legend (e.g., a printed label or insert or other medium describing the product's use (e.g., an audio- or videotape)). The legend can be associated with the container (e.g., affixed to the container) and can describe the manner in which the compositions therein should be administered (e.g., the frequency and route of administration), indications therefor, and other uses. The compositions can be ready for administration (e.g., present in dose-appropriate units), and may include one or more additional pharmaceutically acceptable adjuvants, carriers or other diluents and/or an additional therapeutic agent. Alternatively, the compositions can be provided in a concentrated form with a diluent and instructions for dilution.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### EXAMPLES

Previous studies aiming either at monitoring the effects of an inhibitor of NAD synthesis (as an anti-cancer treatment) on NAD levels in mouse tissue, or at investigating changes in human plasma NAD after intravenous infusion of NAD used tandem mass spectrometry (LC-MS/MS)[57, 58]. However, this assay requires complex and costly instrumentation not widely available with limited applicability for biomarker detection in the clinical setting. In contrast, the NAD detection method described herein, allows quantification of NAD levels in tissue and body fluids using easily accessible laboratory equipment and commercially available reagents, is analytically robust with easy and rapid implementation.

Moreover, NAD is a labile metabolite that is the substrate for multiple enzymes such as NAD hydrolases, ADP ribosylation enzymes and deacetylases[59]. Our method includes sample preparation steps that prevent NAD degradation and artifactual variability in NAD quantitation.

No evidence of reduced NAD levels in tissue, CSF or blood of humans or animals affected by a neurodegenerative disease has been described prior to this invention. Provided herein, is the first evidence of reduced brain NAD levels in an animal model of ALS, opening the path to use NAD quantification as a diagnostic test. Also shown herein for the first time is NAD restoration in the target organ after treatment with a neuroprotective drug. Finally, it is shown that NAD levels are elevated in CSF and blood in an animal model of ALS; this elevation is hypothesized to result from the destruction of brain neurons. The advantage of using NAD as measure of treatment efficacy is that NAD levels are relevant to the pathogenesis of neurodegenerative diseases, therefore normalized NAD levels are indicative of the disease-modifying effect of a treatment. In Parkinson's disease for example where efficient symptomatic treatments are available (dopamine replacement therapy or dopamine agonists), differentiating a symptomatic effect (correction of some symptoms without slowing down neuronal death) over a disease-modifying effect (slower clinical impairment or clinical improvement due to slowing down neuronal death) of a new drug candidate is a challenge necessitating complex clinical trial designs sometimes difficult to implement[27]. Using CSF and/or blood and/or other biosample NAD as a biomarker indicative of improved neuronal health will provide an indication of the disease-modifying effect of the treatment under study.

### Example 1: NAD Quantification Method in Brain Tissue, CSF and Blood

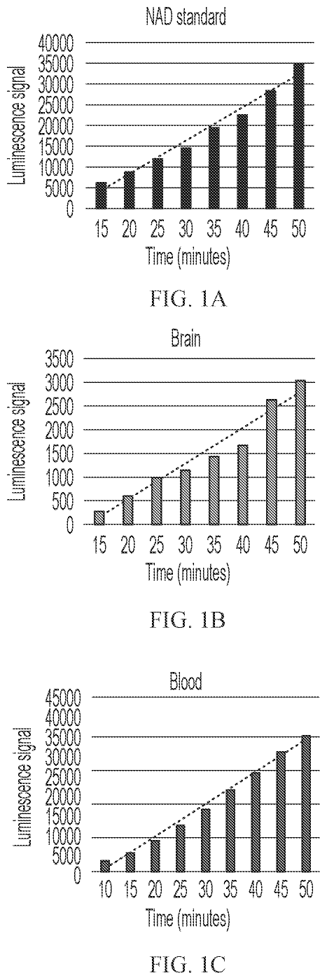

A method for reproducible NAD quantification in brain and blood was developed, requiring only routine laboratory equipment, such that it can be easily performed in clinical laboratories. FIGS. 1A-1J describe the different steps of the method and show a linear increase of the NAD signal between 15 and 50 minutes in murine brain and blood and between 20 and 60 minutes in murine and human CSF. A standard consisting of the metabolite NAD was run in each experiment and showed NAD concentrations of ˜270 nM in murine brain, ˜60 μM in murine whole blood, ˜300 nM in murine CSF and ˜10 nM in human CSF.

Week-to-week reproducibility measured with an NAD standard curve 4 weeks in a row showed inter-assay coefficients of variation (CV %) between 8.4 and 13.7% (Table 2). Intra-assay CV was ≤5%.

TABLE 2
Week-to-week reproducibility of NAD measurements

| NAD | 0.25 μM | 0.5 μM | 1.0 μM |
|---|---|---|---|
| Week 1 | 37,751 | 72,496 | 128,498 |
| Week 2 | 34,103 | 68,211 | 138,945 |
| Week 3 | 28,038 | 58,231 | 114,045 |
| Week 4 | 29,460 | 60,702 | 121,444 |
| CV (%) | 13.7 | 10.2 | 8.4 |

### Example 2: NAD Measurement as a Diagnostic Biomarker in the Brains of ALS Mice

NAD was measured in the brains of Tg (SOD1*G93A) ALS mice[60, 61]. Tg (SOD1*G93A) mice express mutant SOD1 that accounts for 20% of people with familial ALS (fPALS) and is also found in 1-2% of apparently sporadic people with ALS (PALS). Misfolded SOD1 is sometimes found in PALS even in the absence of a mutation[62]. Tg (SOD1*G93A) mice have motor deficits detectable with the rotarod or hanging-wire test from about 75 days of age, and develop clinical signs from about 100 days of age (thinning, then hindlimb paresis). An ALS mouse clinical scale (ALSMCS) was established, defined as follows: 1: no observable impairment; 2: motor deficit detectable in either the hanging wire or the rotarod test; 3: abdominal thinning observed by an experienced investigator; 4: hindlimb paresis; 5: clinical endpoint (hindlimb paralysis, mice unable to raise to reach food).

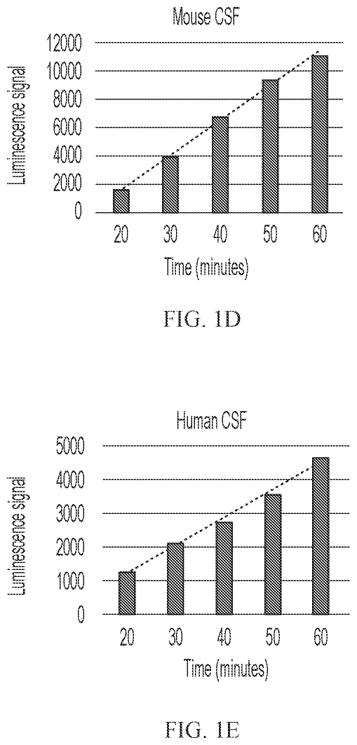

NAD was measured in the brains of three ALS mice cohorts that had been euthanized at ALSMCS 4 and sampled along with age-matched congenic controls (total number of mice examined: 21 ALS, 18 controls). NAD levels were reproducibly reduced by an average of 40% (FIG. 2A shows result of the third cohort). There were no sex-related differences. A fourth cohort was sampled at ALSMCS 5 and NAD levels were found to be reduced by an average of 57% (FIG. 2B). These data strongly suggest a correlation between the extent of brain NAD decrease and disease progression.

### Example 3: Measurement as a Biomarker for Neuroprotection in Brain

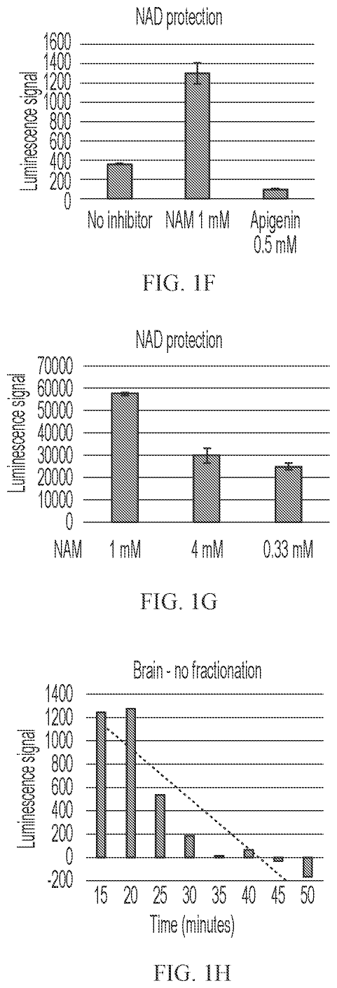

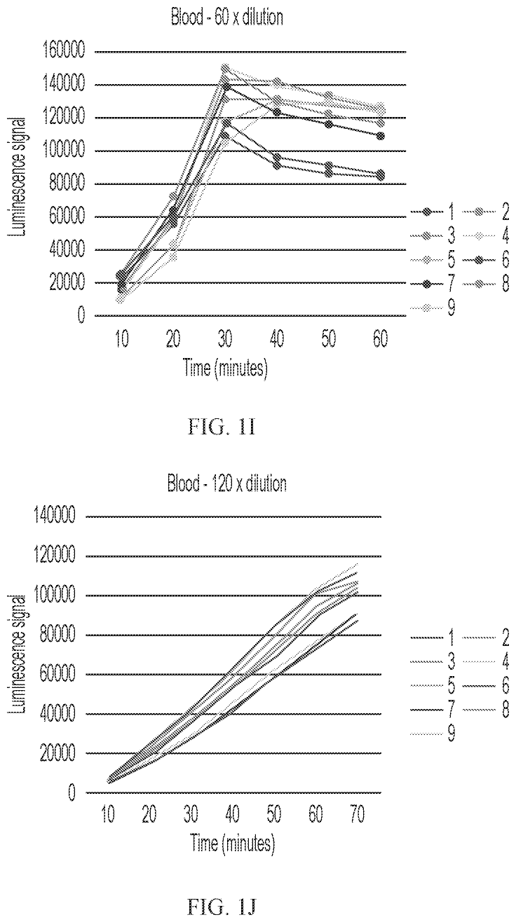

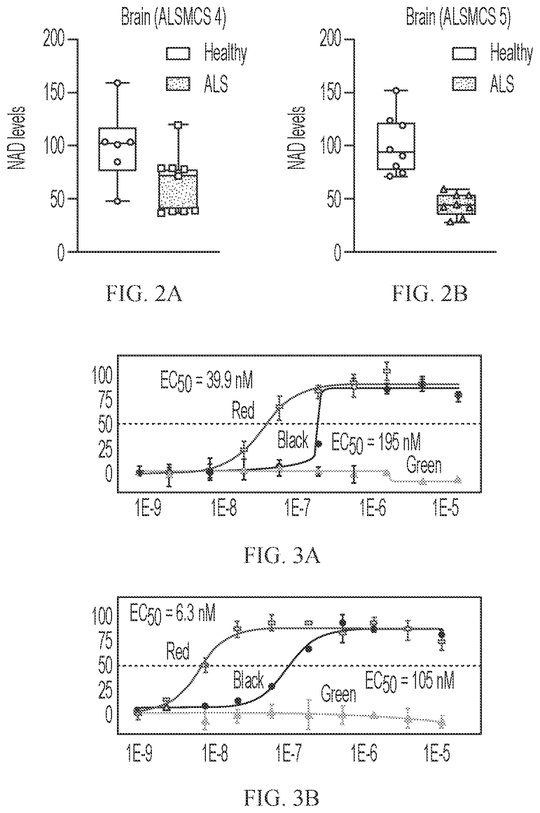

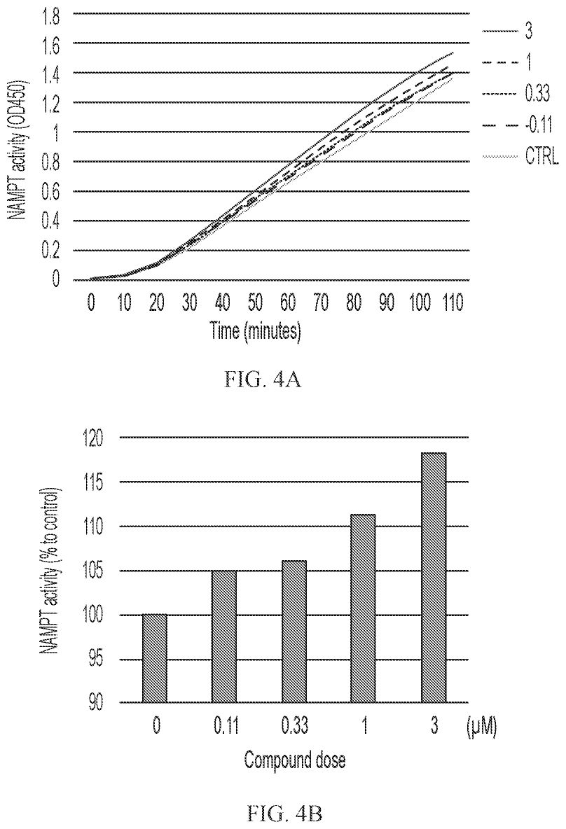

NAD measurement can be used to assess the effect of treatment with compounds elevating NAD by reducing NAD consumption or increasing NAD synthesis, as shown in the example below. SR1457 and its analog SR005 (PCT/US20/32903) are neuroprotective compounds preventing cell death due to misfolded proteins such as TPrP and misfolded alpha-synuclein (involved in PD), and increasing NAD levels by activation of NAMPT, the rate limiting enzyme in NAD synthesis (FIGS. 3A, 3B, 4A and 4B). Treatment of ALS mice with SR005 leads to a significant clinical effect with a delay in the progression of motor impairment (FIG. 5). In SR005 treated ALS mice, increased brain NAD levels demonstrate target engagement by the compound (FIG. 6).

### Example 4: NAD Measurement as a Diagnostic Biomarker in the CSF and Blood of Als Mice

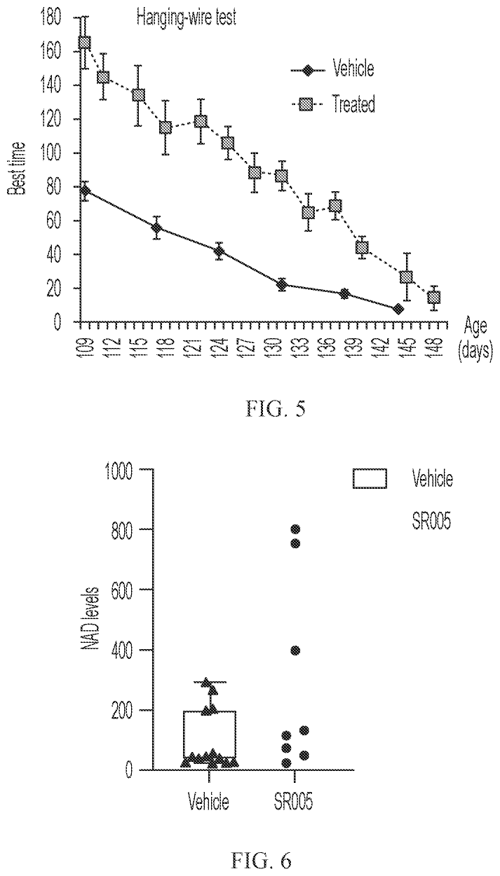

NAD was measured in the CSF and blood of sick Tg (SOD1*G93A) ALS mice. An average increase of CSF NAD by 48% (FIG. 7A, mice at ALSMCS 5) and blood NAD by 23% (FIG. 7B, mice at ALSMCS4) was observed compared to age-matched control mice.

### Example 5: NAD Measurement as a Pharmacodynamic Biomarker in Blood

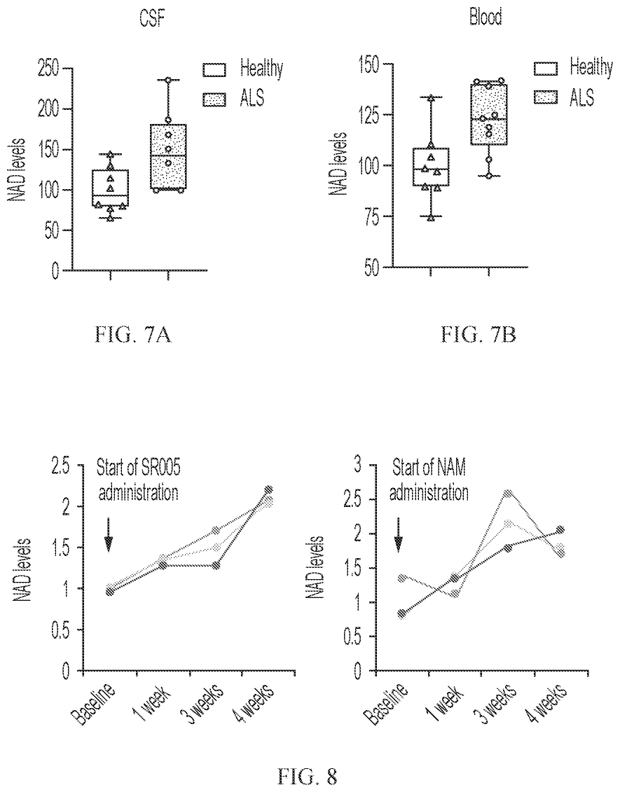

Increased blood NAD levels after treatment of mice with SR005 or nicotinamide (NAM), a precursor of NAD in the salvage synthesis pathway, show the use of NAD measurements in blood as a pharmacodynamic biomarker (FIG. 8).

In the present disclosure, an easy-to-implement and analytically robust method for the detection of NAD in brain tissue, CSF and blood is shown. A progressive reduction of NAD levels in the brains of ALS mice correlating with disease progression, and elevation of brain NAD levels in mice correlating with clinical efficacy of a neuroprotective compound was found. An increase in NAD levels in CSF and blood of sick ALS mice was also found. It is hypothesized that this increase in CSF and blood be due to the release of intracellular NAD by damaged neurons. We expect NAD levels in tissue and body fluids to be normalized upon treatment with a neuroprotective compound. Finally, it was shown that the pharmacodynamic effect of an NAD elevating treatment can be monitored by quantification of NAD in blood. Therefore, NAD measurement in CSF, blood and other tissues or body fluids has utility in diagnosing ALS and other neurodegenerative, degenerative and metabolic conditions where NAD metabolism is compromised. Since NAD levels are a molecular signature of cellular demise that occurs progressively in these diseases, it is proposed herein that NAD quantification in biosamples is a prognostic biomarker and a biomarker for disease progression. It was shown that NAD levels can be pharmacologically modulated in blood and the CNS, therefore tissue and/or biofluid NAD measurement can be used as a pharmacodynamic biomarker to evaluate the disease-modifying effect of an investigational drug candidate in clinical trials and as an outcome measure of treatment efficacy.

### REFERENCES

1 Westeneng, H. J., et al., Prognosis for patients with amyotrophic lateral sclerosis: development and validation of a personalised prediction model. Lancet Neurol, 2018. 17(5): p. 423-433.
2. Masrori, P. and P. Van Damme, Amyotrophic lateral sclerosis: a clinical review. Eur J Neurol, 2020.
3. Ghasemi, M. and R. H. Brown, Jr., Genetics of Amyotrophic Lateral Sclerosis. Cold Spring Harb Perspect Med, 2018. 8(5).
4. Rosen, D. R., et al., Mutations in Cu/Zn superoxide dismutase gene are associated with familial amyotrophic lateral sclerosis. Nature, 1993. 362(6415): p. 59-62.
5. Deng, H. X., et al., Amyotrophic lateral sclerosis and structural defects in Cu,Zn superoxide dismutase. Science, 1993. 261(5124): p. 1047-51.
6. Lattante, S., G. A. Rouleau, and E. Kabashi, TARDBP and FUS mutations associated with amyotrophic lateral sclerosis: summary and update. Hum Mutat, 2013. 34(6): p. 812-26.
7 Dawson, T. M. and V. L. Dawson, Rare genetic mutations shed light on the pathogenesis of Parkinson disease. J Clin Invest, 2003. 111(2): p. 145-51.
8. Farrer, M. J., Genetics of Parkinson disease: paradigm shifts and future prospects. Nat Rev Genet, 2006. 7(4): p. 306-18.
9. Klein, C. and M. G. Schlossmacher, Parkinson disease, 10 years after its genetic revolution: multiple clues to a complex disorder. Neurology, 2007. 69(22): p. 2093-104.
10. Pankratz, N., et al., Genomewide association study for susceptibility genes contributing to familial Parkinson disease. Hum Genet, 2009. 124(6): p. 593-605.
11. Martin, I., V. L. Dawson, and T. M. Dawson, Recent advances in the genetics of Parkinson's disease. Annu Rev Genomics Hum Genet, 2011. 12: p. 301-25.
12. Zhao, F., et al., Mutations of glucocerebrosidase gene and susceptibility to Parkinson's disease: An updated meta-analysis in a European population. Neuroscience, 2016. 320: p. 239-46.
13. Goyal, N. A., et al., Addressing heterogeneity in amyotrophic lateral sclerosis CLINICAL TRIALS. Muscle Nerve, 2020.
14. van Eijk, R. P. A., et al., Refining eligibility criteria for amyotrophic lateral sclerosis clinical trials. Neurology, 2019.
15. Berry, J. D., et al., Improved stratification of ALS clinical trials using predicted survival. Ann Clin Transl Neurol, 2018. 5(4): p. 474-485.
16. Benatar, M., et al., Validation of serum neurofilaments as prognostic and potential pharmacodynamic biomarkers for ALS. Neurology, 2020.
17. Huang, F., et al., Longitudinal biomarkers in amyotrophic lateral sclerosis. Ann Clin Transl Neurol, 2020.
18. Gaiani, A., et al., Diagnostic and Prognostic Biomarkers in Amyotrophic Lateral Sclerosis: Neurofilament Light Chain Levels in Definite Subtypes of Disease. JAMA Neurol, 2017. 74(5): p. 525-532.
19. Shepheard, S. R., et al., The extracellular domain of neurotrophin receptor p75 as a candidate biomarker for amyotrophic lateral sclerosis. PLoS One, 2014. 9(1): p. e87398.
20. Matusica, D., et al., Inhibition of motor neuron death in vitro and in vivo by a p75 neurotrophin receptor intracellular domain fragment. J Cell Sci, 2016. 129(3): p. 517-30.
21. Shepheard, S. R., et al., Urinary p75 (ECD): A prognostic, disease progression, and pharmacodynamic biomarker in ALS. Neurology, 2017. 88(12): p. 1137-1143.
22. Majumder, V., et al., TDP-43 as a potential biomarker for amyotrophic lateral sclerosis: a systematic review and meta-analysis. BMC Neurol, 2018. 18(1): p. 90.
23. Martinez, H. R., et al., Increased cerebrospinal fluid levels of cytokines monocyte chemoattractant protein-I (MCP-1) and macrophage inflammatory protein-1 beta (MIP-1 beta) in patients with amyotrophic lateral sclerosis. Neurologia, 2020. 35(3): p. 165-169.
24. Sun, J., et al., Blood biomarkers and prognosis of amyotrophic lateral sclerosis. Eur J Neurol, 2020.
25. Vu, L. T. and R. Bowser, Fluid-Based Biomarkers for Amyotrophic Lateral Sclerosis. Neurotherapeutics, 2017. 14(1): p. 119-134.
26. Freischmidt, A., et al., Systemic dysregulation of TDP-43 binding microRNAs in amyotrophic lateral sclerosis. Acta Neuropathol Commun, 2013. 1: p. 42.
27. Athauda, D. and T. Foltynie, Challenges in detecting disease modification in Parkinson's disease clinical trials. Parkinsonism Relat Disord, 2016. 32: p. 1-11.
28. Soderbom, G., Status and future directions of clinical trials in Parkinson's disease. Int Rev Neurobiol, 2020. 154: p. 153-188.
29. Nakajima, A., et al., Dopamine transporter imaging predicts motor responsiveness to levodopa challenge in patients with Parkinson's disease: A pilot study of DATSCAN for subthalamic deep brain stimulation. J Neurol Sci, 2018. 385: p. 134-139.
30. Parnetti, L., et al., CSF and blood biomarkers for Parkinson's disease. Lancet Neurol, 2019. 18(6): p. 573-586.
31. Forsberg Moren, A. and A. Varrone, Timing is everything: tau imaging across stages of Alzheimer's disease. Brain, 2020. 143(9): p. 2634-2636.
32. Kim, J. S., Tau Imaging: New Era of Neuroimaging for Alzheimer's Disease. Nucl Med Mol Imaging, 2020. 54(4): p. 161-162.
33. Hansson, O., et al., Advantages and disadvantages of the use of the CSF Amyloid beta (Abeta) 42/40 ratio in the diagnosis of Alzheimer's Disease. Alzheimers Res Ther, 2019. 11(1): p. 34.
34. Thijssen, E. H., et al., Diagnostic value of plasma phosphorylated tau181 in Alzheimer's disease and frontotemporal lobar degeneration. Nat Med, 2020. 26(3): p. 387-397.
35. Palmqvist, S., et al., Discriminative Accuracy of Plasma Phospho-tau217 for Alzheimer Disease vs Other Neurodegenerative Disorders. JAMA, 2020. 324(8): p. 772-781.
36. Escande, C., et al., Flavonoid apigenin is an inhibitor of the NAD+ ase CD38: implications for cellular NAD+ metabolism, protein acetylation, and treatment of metabolic syndrome. Diabetes, 2013. 62(4): p. 1084-93.
37. Zhou, M., et al., Highly neurotoxic monomeric alpha-helical prion protein. Proc Natl Acad Sci USA, 2012. 109(8): p. 3113-8.
38. Houtkooper, R. H., et al., The secret life of NAD+: an old metabolite controlling new metabolic signaling pathways. Endocr Rev, 2010. 31(2): p. 194-223.
39. Zhou, M., et al., Neuronal death induced by misfolded prion protein is due to NAD+ depletion and can be relieved in vitro and in vivo by NAD+ replenishment. Brain, 2015. 138(4): p. 992-1008.
40. Lehmann, S., S. H. Loh, and L. M. Martins, Enhancing NAD+ salvage metabolism is neuroprotective in a PINK1 model of Parkinson's disease. Biol Open, 2016. 6: p. 141-147.
41. Harlan, B. A., et al., Evaluation of the NAD(+) biosynthetic pathway in ALS patients and effect of modulating NAD(+) levels in hSOD1-linked ALS mouse models. Exp Neurol, 2020. 327: p. 113219.
42. Sorrentino, V., et al., Enhancing mitochondrial proteostasis reduces amyloid-beta proteotoxicity. Nature, 2017. 552(7684): p. 187-193.
43. Hou, Y., et al., NAD(+) supplementation normalizes key Alzheimer's features and DNA damage responses in a new AD mouse model with introduced DNA repair deficiency. Proc Natl Acad Sci USA, 2018. 115(8): p. E1876-E1885.
44. Zhang, H., et al., NAD(+) repletion improves mitochondrial and stem cell function and enhances life span in mice. Science, 2016. 352(6292): p. 1436-43.
45. Mouchiroud, L., et al., The NAD(+)/Sirtuin Pathway Modulates Longevity through Activation of Mitochondrial UPR and FOXO Signaling. Cell, 2013. 154(2): p. 430-41.
46. Penberthy, W. T. and I. Tsunoda, The importance of NAD in multiple sclerosis. Curr Pharm Des, 2009. 15(1): p. 64-99.
47. Brown, K. D., et al., Activation of SIRT3 by the NAD(+) precursor nicotinamide riboside protects from noise-induced hearing loss. Cell Metab, 2014. 20(6): p. 1059-68
48. Lin, J. B., et al., NAMPT-Mediated NAD(+) Biosynthesis Is Essential for Vision In Mice. Cell Rep, 2016. 17(1): p. 69-85.
49. Satchell, M. A., et al., A dual role for poly-ADP-ribosylation in spatial memory acquisition after traumatic brain injury in mice involving NAD+ depletion and ribosylation of 14-3-3gamma. J Neurochem, 2003. 85(3): p. 697-708.
50. Vaur, P., et al., Nicotinamide riboside, a form of vitamin B3, protects against excitotoxicity-induced axonal degeneration. FASEB J, 2017. 31(12): p. 5440-5452.
51. Ralto, K. M., E. P. Rhee, and S. M. Parikh, NAD(+) homeostasis in renal health and disease. Nat Rev Nephrol, 2020. 16(2): p. 99-111.
52. Ying, W., et al., Intranasal administration with NAD+ profoundly decreases brain injury in a rat model of transient focal ischemia. Front Biosci, 2007. 12: p. 2728-34.
53. Hsu, C. P., et al., Nicotinamide phosphoribosyltransferase regulates cell survival through NAD+ synthesis in cardiac myocytes. Circ Res, 2009. 105(5): p. 481-91.
54. Yamamoto, T., et al., Nicotinamide mononucleotide, an intermediate of NAD+ synthesis, protects the heart from ischemia and reperfusion. PLoS One, 2014. 9(6): p. e98972.
55. Yoshino, J., et al., Nicotinamide mononucleotide, a key NAD(+) intermediate, treats the pathophysiology of diet- and age-induced diabetes in mice. Cell Metab, 2011. 14(4): p. 528-36.
56. Trammell, S. A., et al., Nicotinamide Riboside Opposes Type 2 Diabetes and Neuropathy in Mice. Sci Rep, 2016. 6: p. 26933.
57. Liang, X., et al., Measuring NAD(+) levels in mouse blood and tissue samples via a surrogate matrix approach using LC-MS/MS. Bioanalysis, 2014. 6(11): p. 1445-57.
58. Grant, R., et al., A Pilot Study Investigating Changes in the Human Plasma and Urine NAD+ Metabolome During a 6 Hour Intravenous Infusion of NAD. Front Aging Neurosci, 2019. 11: p. 257.
59. Stromland, O., et al., Keeping the balance in NAD metabolism. Biochem Soc Trans, 2019. 47(1): p. 119-130.
Gurney, M. E., et al., Motor neuron degeneration in mice that express a human Cu,Zn superoxide dismutase mutation. Science, 1994. 264(5166): p. 1772-5.
61. Scott, S., et al., Design, power, and interpretation of studies in the standard murine model of ALS. Amyotroph Lateral Scler, 2008. 9(1): p. 4-15.
62. Pokrishevsky, E., et al., Aberrant localization of FUS and TDP43 is associated with misfolding of SOD I in amyotrophic lateral sclerosis. PLoS One, 2012. 7(4): p. e35050.

### OTHER EMBODIMENTS

From the foregoing description, it will be apparent that variations and modifications may be made to the invention described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

What is claimed:

1. A method of detecting and quantifying nicotinamide dinucleotide (NAD) in a biological sample, comprising:
protecting NAD in the biological sample using an inhibitor of NAD degradation;
optionally disrupting the biological sample;
optionally clarifying the biological sample to obtain a clarified sample;
optionally fractionating the biological sample;
collecting the biological sample; and
measuring NAD;
thereby, detecting and quantifying nicotinamide dinucleotide (NAD) in the biological sample.

2. The method of claim 1, wherein the inhibitor of NAD degradation comprises a metabolite, a chemical reagent, a chemical compound, a lipid, a polymer, a nucleic acid or a protein.

3. The method of claim 2, wherein the inhibitor of NAD degradation comprises nicotinamide.

4. The method of claim 1, wherein the addition of the inhibitor of NAD degradation occurs at the same time as diluting the biological sample.

5. The method of claim 1, wherein the biological sample is disrupted by mechanical or chemical lysis and/or sonication.

6. The method of claim 1, wherein the biological sample is clarified by centrifugation and/or filtration.

7. The method of claim 1, wherein the fractionating is performed by filtration and/or chromatography.

8. The method of claim 1, wherein the fractionated biological sample comprises a fraction cut of about 100 kDa, 50 kDa, 30 kDa, 10 kDa, 5 kDa or 3 kDa.

9. The method of claim 1, wherein the fractionating removes one or more factors interfering with NAD detection.

10. The method of claim 1, wherein the measuring the NAD comprises measuring and quantifying NAD metabolite levels using an enzymatic cycling assay.

11. A method of diagnosing and/or monitoring disease progression in a subject likely to develop or having a neurodegenerative, degenerative or metabolic disease using the method of claim 1.

12. A method of preventing or treating a subject likely to develop or having a neurodegenerative, degenerative or metabolic disease comprising;
diagnosing the subject using a method of claim 1; and,
administering to the subject a therapeutic agent;
thereby, preventing disease onset or treating the subject having a neurodegenerative, degenerative or metabolic disease.

13. The method of claim 11 or 12, wherein the neurodegenerative disease comprises Alzheimer's disease (AD), Parkinson's diseases (PD), dementia with Lewy Bodies (DLB), Fronto-temporal dementia (FTD), chronic traumatic encephalopathy (CTE), prion diseases, Huntington's disease (HD), amyotrophic lateral sclerosis (ALS) or multiple sclerosis (MS).

14. The method of claim 11 or 12, wherein the metabolic disease is diabetes or non-alcoholic fatty liver disease (NAFLD).

15. The method of claim 12, wherein the pharmacological effect of the treatment is monitored by quantification of nicotinamide dinucleotide (NAD) in blood, brain, cerebrospinal fluid, another biological fluid or a tissue.

16. The method of claim 15, wherein a decrease or increase in NAD as compared to a normal control, is diagnostic of a neurodegenerative, degenerative or metabolic disease.

17. The method of claim 15, wherein detection of an increase or decrease in NAD or normalization of NAD levels as compared to a normal control, is indicative of therapeutic efficacy of the treatment.

18. A method of identifying candidate neuroprotective or disease-modifying therapeutic agents comprising contacting a cell or biological sample with a candidate agent, measuring the nicotinamide dinucleotide (NAD) levels in the cell or biological samples as compared to controls and determining whether NAD is modulated in response to the agent.

19. The method of claim 18, wherein an increase in NAD levels as compared to a control is indicative of a neuroprotective and/or disease-modifying therapeutic agent.

20. The method of claim 18, wherein the cells comprise cells obtained from a subject diagnosed with a neurodegenerative, degenerative or metabolic disorder, cell lines, transfected cells, mesenchymal cells, induced pluripotent cells (iPSCs), stem cells or combinations thereof.

21. The method of claim 18, wherein the assay is a high-throughput screening assay.
